(19) United States
(12) Patent Application Publication
(10) Pub. No.: US 2022/0378988 A1
(43) Pub. Date: Dec. 1, 2022

(54) BIOLOGICAL COMPONENT CASSETTE, BIOLOGICAL COMPONENT KIT, AND BIOLOGICAL COMPONENT TREATMENT SYSTEM

(71) Applicants: TERUMO KABUSHIKI KAISHA, TOKYO (JP); TERUMO BCT, INC., LAKEWOOD, CO (US)

(72) Inventors: Masatsugu IGARASHI, Shizuoka (JP); Briden STANTON, Highlands Ranch, CO (US); Jon DODD, Littleton, CO (US)

(21) Appl. No.: 17/886,038

(22) Filed: Aug. 11, 2022

Related U.S. Application Data
(63) Continuation of application No. PCT/JP2021/004746, filed on Feb. 9, 2021.

(30) Foreign Application Priority Data
Feb. 14, 2020 (JP) JP2020-023405

(51) Int. Cl. A61M 1/02; C12M 1/00; C12M 3/00

(52) U.S. Cl. CPC A61M 1/0272; C12M 23/14; C12M 23/26; C12M 23/42; A61M 1/0281; A61M 2205/12; A61M 2205/3331

(57) ABSTRACT

A biological component cassette includes a cassette main body having a flow path for the liquid in an interior of the cassette main body. The cassette main body is flexible and has a sheet-like shape. The biological component cassette includes a frame that is less flexible than the cassette main body. The frame includes an accommodation chamber in which the cassette main body is accommodated. A side portion of the frame forms at least part of the accommodation chamber. When the cassette main body is accommodated in the accommodation chamber, one surface of the cassette main body is covered by a bottom portion of the accommodation chamber, and another surface of the cassette main body is exposed to an external environment.

### CROSS REFERENCE TO RELATED APPLICATIONS

This application is a continuation of and claims priority to PCT Application No. PCT/JP2021/004746 filed on Feb. 9, 2021, entitled “CELL WASH CASSETTE” which claims priority to Japanese Patent Application No. 2020-023405 filed on Feb. 14, 2020. The entire disclosures of the applications listed above are hereby incorporated herein by reference, in their entireties, for all that they teach and for all purposes.

### BACKGROUND

The present disclosure relates to a biological component cassette having flow paths through which a biological component flows, a biological component kit including the biological component cassette, and a biological component treatment system including the biological component kit and the biological component treatment device.

In the practice of regenerative medicine, biological cells (biological components) are collected and cultured, and the cultured cells are administered to a patient. In such a regenerative medicine process, before the cells are administered to the patient, a cleaning treatment is performed to remove (or clean) a culture medium (or culture solution) or other foreign substances and to increase the cell concentration. In the cleaning treatment, for example, a cell cleaning device as disclosed in Japanese Laid-Open Patent Publication No. 2015-188315 is used.

### SUMMARY

In related art cleaning treatments, a path through which the culture is supplied, a path through which a cleaning solution is supplied, a path over which the culture is treated, a path by which cells (products) after cleaning are extracted, and the like are interconnected in accordance with settings of the cell cleaning device, and liquids are circulated in an appropriate order under the operation of the cell cleaning device. In the related art, an operator must set the tubes that form the respective paths in the cell cleaning device, which makes the cleaning treatment time consuming and complicated.

In some cases, in order to improve the working efficiency of the cleaning treatment, a configuration may be considered in which a plurality of paths are integrated in a rigid biological component cassette, and the biological component cassette is set in the cell cleaning device. However, when such a rigid biological component cassette is applied, it is difficult for the flow paths in the interior to be deformed, and a mechanism or the like for detecting a state (for example, the pressure) of the liquid in the flow paths must be provided on an external part of the cassette. In this case, in addition to setting the biological component cassette, the operator sets detection channel portions on the external part, which consumes additional time and reduces efficiency.

The present disclosure has been devised taking into consideration the aforementioned problems, and provides a biological component cassette, a biological component kit, and a biological component treatment system which are capable of enhancing working efficiency while enabling an operator to set a plurality of paths more easily. In addition, the present disclosure provides deformable flow paths.

A first aspect of the present disclosure comprises a biological component cassette configured to allow a liquid containing a biological component to flow, the biological component cassette comprising a cassette main body having a flow path for the liquid in an interior of the cassette main body, and which is formed into a flexible sheet-like shape, and a frame made less flexible than the cassette main body and retaining the cassette main body, wherein the frame includes an accommodation chamber in which the cassette main body is accommodated, and a side portion forming the accommodation chamber, and one surface of the cassette main body is covered by a bottom portion of the accommodation chamber, and another surface of the cassette main body is exposed from the frame.

A second aspect of the present disclosure comprises a biological component kit having a tube configured to allow a liquid containing a biological component to flow, and a biological component cassette to which the tube is connected, wherein the biological component cassette comprises a cassette main body having a flow path for the liquid in an interior of the cassette main body, and which is formed into a flexible sheet-like shape, and a frame made less flexible than the cassette main body and retaining the cassette main body, wherein the frame includes an accommodation chamber in which the cassette main body is accommodated, and a side portion forming the accommodation chamber, and one surface of the cassette main body is covered by a bottom portion of the accommodation chamber, and another surface of the cassette main body is exposed from the frame.

A third aspect of the present disclosure comprises a biological component treatment system comprising a biological component kit having a tube configured to allow a liquid containing a biological component to flow, and a biological component cassette to which the tube is connected, and a biological component treatment device in which the biological component kit is set, wherein the biological component cassette comprises a cassette main body having a flow path for the liquid in an interior of the cassette main body, and which is formed into a flexible sheet-like shape, and a frame made less flexible than the cassette main body and retaining the cassette main body, wherein the frame includes an accommodation chamber in which the cassette main body is accommodated, and a side portion forming the accommodation chamber, and one surface of the cassette main body is covered by a bottom portion of the accommodation chamber, and another surface of the cassette main body is exposed from the frame, and the biological component treatment device includes a cassette setting location in which the biological component cassette is set.

In at least one example of the above-described biological component cassette, the biological component kit, and the biological component treatment system, the cassette main body which possesses flexibility is retained by the rigid frame. Consequently, a user can easily set a plurality of paths which are used for treatment of the biological component in the biological component treatment device, and it is possible to enhance working efficiency. In addition, in the biological component cassette, the biological component kit, and the biological component treatment system, by the side portion exposing one surface of the cassette main body, and the cassette main body which possesses flexibility constituting the flow paths, operations of deforming the flow paths or the like can be easily performed with respect to the cassette main body, and the state of the liquid in the flow paths can be suitably detected with high accuracy.

### BRIEF DESCRIPTION OF THE DRAWINGS

FIG. 1 is a perspective view showing a biological component treatment system including a biological component cassette and a biological component kit according to at least one embodiment of the present disclosure;

FIG. 2 is an exploded perspective view of the biological component cassette according to at least one embodiment of the present disclosure;

FIG. 3 is a partially enlarged perspective view of a cassette main body and a frame according to at least one embodiment of the present disclosure;

FIG. 4 is an explanatory diagram showing liquid paths of the biological component kit and flow paths of the cassette main body according to at least one embodiment of the present disclosure;

FIG. 5A is a partial cross-sectional view showing a pressure detector of the biological component treatment system according to at least one embodiment of the present disclosure;

FIG. 5B is a partial cross-sectional view showing operations when a liquid flows through a detection channel portion according to at least one embodiment of the present disclosure;

FIG. 6 is a perspective view of a gripper member according to at least one embodiment of the present disclosure;

FIGS. 7A and 7B are perspective views showing the gripper member according to at least one embodiment of the present disclosure;

FIG. 8 is a first operation diagram showing operations of the biological component system according to at least one embodiment of the present disclosure;

FIG. 9 is a second operation diagram showing operations of the biological component system according to at least one embodiment of the present disclosure;

FIG. 10 is a third operation diagram showing operations of the biological component system according to at least one embodiment of the present disclosure;

FIG. 11 is a fourth operation diagram showing operations of the biological component system according to at least one embodiment of the present disclosure; and

FIG. 12 is a perspective view showing a biological component treatment system including a biological component cassette and a biological component kit according to another embodiment of the present disclosure.

### DETAILED DESCRIPTION

Example embodiments of the present disclosure are presented and described in detail below with reference to the accompanying drawings.

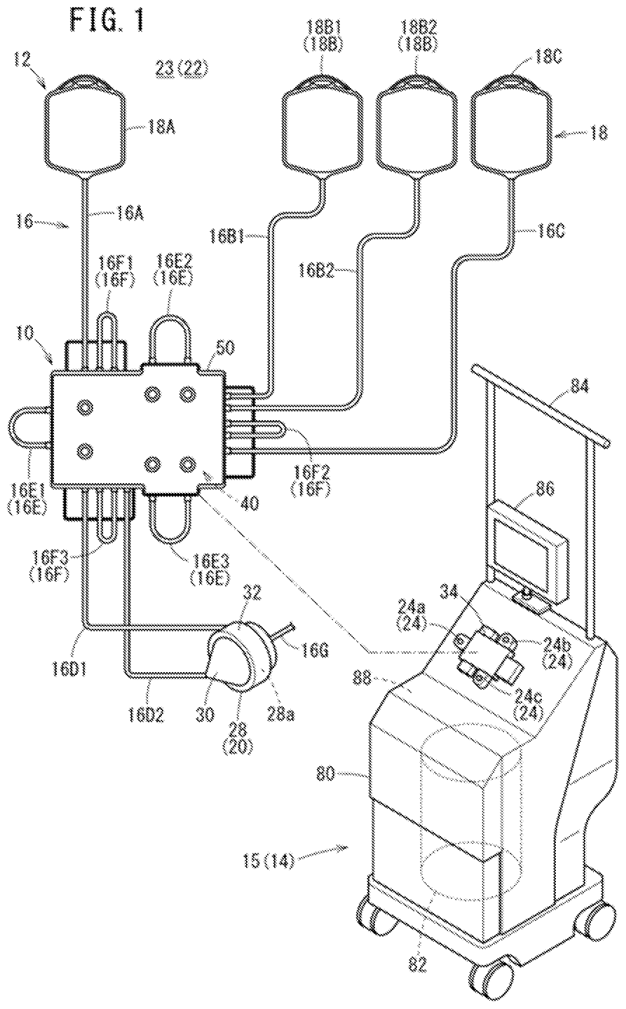

A biological component cassette 10 (hereinafter, simply referred to as a cassette 10) according to an embodiment of the present disclosure, as shown in FIG. 1, forms one part of a biological component kit 12 (hereinafter, simply referred to as a kit 12), and is set in a biological component treatment device 14. The cassette 10 collects together a plurality of paths of the kit 12, and is used as a structural body through which a liquid containing a biological component and a liquid for treatment of the biological component are capable of flowing (being circulated).

The kit 12 includes, as members that form a plurality of paths, and in addition to the cassette 10, a plurality of tubes 16, a plurality of medical bags 18, and a treatment unit 20 to be treated in the biological component treatment device 14. The kit 12 allows a plurality of types of liquids contained in each of the medical bags 18 to flow through the cassette 10 and through each of the tubes 16 under the operation of the biological component treatment device 14, and is formed so as to obtain a target product by treating the liquids in the treatment unit 20.

The kit 12 according to the present embodiment is configured as a cell cleaning kit, and includes a culture having cells (biological components) of a living body, and a culture medium or a preservative solution in the form of a liquid flowing through the interior thereof, and a cleaning solution for cleaning the cells. More specifically, the kit 12 and the biological component treatment device 14 are included as part of a biological component treatment system 22 (cell cleaning system 23) applied to a cleaning treatment, which is one step of a regenerative medicine process. The cell cleaning system 23 performs a cleaning treatment for removing the culture medium and the like from the culture having the cultured cells by using the cleaning solution, thereby increasing the cell concentration. Thus, hereinafter, the biological component treatment device 14 may also be referred to as a cell cleaning device 15.

The cells of a living body are not particularly limited, and may include, for example, cells (T cells and the like) contained in blood, and stem cells (ES cells, iPS cells, mesenchymal stem cells, and the like). The cleaning solution may be selected appropriately depending on the cells of the living body, and for example, a buffer solution such as Phosphate Buffered Salts (PBS), Tris-Buffered Saline (TBS), or physiological saline and the like may be used.

The plurality of medical bags 18 include a culture bag 18A in which the culture is accommodated, two cleaning solution bags 18B (a first cleaning solution bag 18B1 and a second cleaning solution bag 18B2) in which the cleaning solution is accommodated, and a product bag 18C for accommodating the cells that have been cleaned.

In a culture step of the regenerative medicine process, the culture bag 18A is placed in a state having the culture, by storing the cells, which have been cultured in the culture medium, in the interior thereof by an appropriate method. On the other hand, the cleaning solution bag 18B is assembled into the kit 12 in a state with the cleaning solution stored therein, and is sealed using a sterile bonding apparatus or the like. Alternatively, the cleaning solution bag 18B may be assembled beforehand in the kit 12 in a state in which the cleaning solution is stored and sealed therein, and the seal may be released at the time of being set. The product bag 18C is connected in an empty state before the cleaning treatment is performed, and the cells (including the cleaning solution) which have been concentrated during the cleaning treatment are stored to become the product. A preservative solution or the like may also be stored beforehand in the product bag 18C.

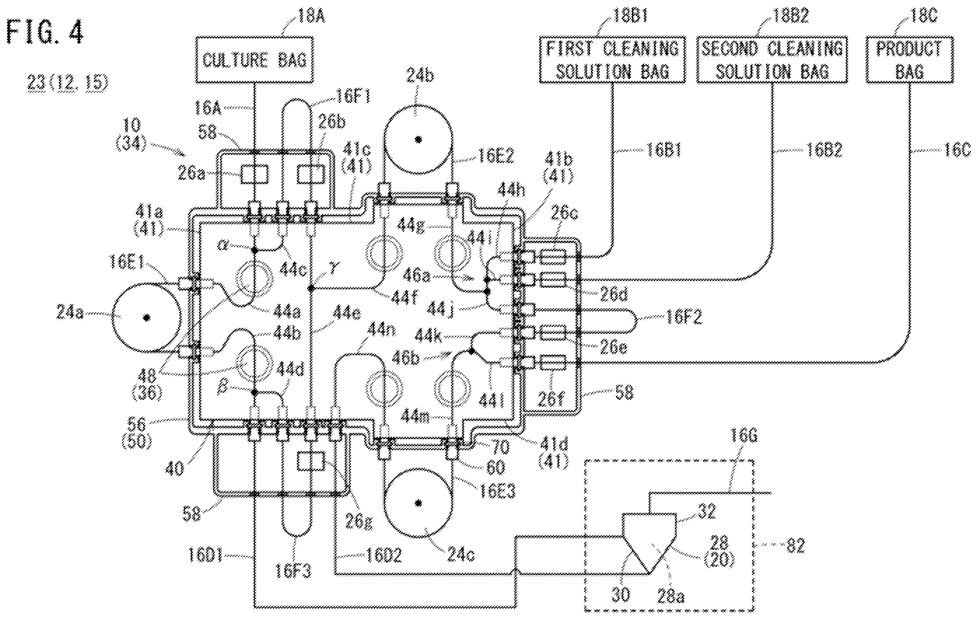

The plurality of tubes 16 of the kit 12 include a culture tube 16A connected between the culture bag 18A and the cassette 10, a first cleaning solution tube 16B1 connected between the first cleaning solution bag 18B1 and the cassette 10, a second cleaning solution tube 16B2 connected between the second cleaning solution bag 18B2 and the cassette 10, a product tube 16C connected between the product bag 18C and the cassette 10, and two tubes 16 (a first treatment tube 16D1 and a second treatment tube 16D2) connected between the treatment unit 20 and the cassette 10. Further, the plurality of tubes 16 include closed tubes 16 that protrude from the cassette 10, and may be folded back and connected to the cassette 10 again. As such tubes 16, there are a plurality of (three) pump tubes 16E that are set in a plurality of (three) pumps 24 of the cell cleaning device 15, and a plurality of (three) clamp tubes 16F that are set within clamps 26 (refer to FIG. 4) for opening and closing the paths of the kit 12. Furthermore, the plurality of tubes 16 includes a disposal tube 16G that communicates with the exterior of the kit 12 (for example, with a non-illustrated disposal unit of the cell cleaning device 15) without intervening from the treatment unit 20 to the cassette 10.

Connecting structures (not shown) that can be connected together are provided on the culture tube 16A and the culture bag 18A, the first cleaning solution tube 16B1 and the first cleaning solution bag 18B1, and the second cleaning solution tube 16B2 and the second cleaning solution bag 18B2. The culture bag 18A, the first cleaning solution tube 16B1, and the second cleaning solution bag 18B2 are connected to the culture tube 16A, the first cleaning solution tube 16B1, and the second cleaning solution tube 16B2 through the connecting structures prior to implementing the cleaning treatment. Consequently, the culture is made capable of flowing out to the flow path of the culture tube 16A, and the cleaning solution is made capable of flowing out to the first cleaning solution tube 16B1 and the second cleaning solution tube 16B2.

The treatment unit 20 of the kit 12 includes a treatment case 28 in which the culture and the cleaning solution are accommodated (e.g., temporarily accommodated. Applying a centrifugal force from the cell cleaning device 15 to the culture, which is accommodated in an internal space 28a, centrifugally separates the culture into the cells and the culture medium. The treatment case 28 is formed in a hollow three-dimensional shape formed by joining a conical portion 30 and a cylindrical portion 32 in an axial direction. In a set state of the cell cleaning device 15, in the treatment case 28, an apex of the conical portion 30 is arranged on a side remote from the center of centrifugation, and a bottom portion of the cylindrical portion 32 is arranged on a side proximate to the center of centrifugation.

When a centrifugal force is applied thereto, the treatment case 28 moves cells having a high specific gravity outward in the centrifugal direction within the tapered internal space 28a, and moves the culture medium having a low specific gravity inward in the centrifugal direction. The first treatment tube 16D1 is connected to a side of the cylindrical portion 32 of the treatment case 28, whereas the second treatment tube 16D2 is connected to the apex of the conical portion 30. Further, the disposal tube 16G is connected to the bottom of the treatment case 28 in order to discard the culture medium that was separated by centrifugation.

In addition, by joining the plurality of tubes 16 in advance, the cassette 10 becomes a relay for the kit 12, which circulates the culture or the cleaning solution in each of the respective medical bags 18 to other medical bags 18 or to the treatment unit 20. When the kit 12 is set in the cell cleaning device 15, the cassette 10 is attached to the cassette setting location 34 of the cell cleaning device 15, which simplifies the tube arrangement operation of the tubes 16 in the cleaning treatment.

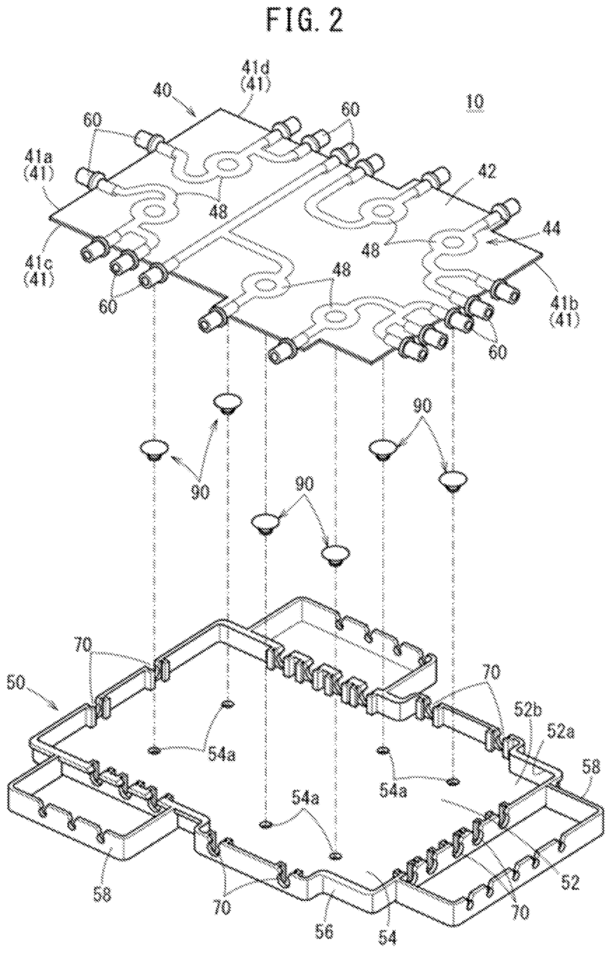

As shown in FIG. 2, the cassette 10 according to the present embodiment includes a soft (or flexible) cassette main body 40 to which the plurality of tubes 16 are directly connected, and a rigid frame 50 that retains the cassette main body 40 and is fixed to the cell cleaning device 15.

The cassette main body 40 exhibits a substantially rectangular shape, and is formed in a thin sheet-like shape which possesses flexibility. The cassette main body 40 may be formed by stacking and joining together two resin sheets 42 made of a resin material in the thickness direction. The resin material constituting the resin sheets 42 is not particularly limited, insofar as the resin possesses flexibility that is capable of being deformed by the pressure of the liquids. Example resins include a vinyl chloride resin, a polyolefin resin, a polyurethane resin, and/or the like may be used.

The cassette main body 40 includes a plurality of flow paths 44 on the inner side thereof, where the flow paths maybe formed by fusion bonding the resin sheets 42 to themselves around protruding flow path walls. An embossing process may be implemented on the surface of the cassette main body 40. A plurality of connectors 60 for connecting the plurality of tubes 16 are provided on outer edges 41 of the cassette main body 40.

On the other hand, the frame 50 includes a resin material that is harder (or less flexible) than the cassette main body 40, and is formed in a thin recessed shape having an accommodation chamber 52 in which the cassette main body 40 is accommodated. The material of the frame 50 is not limited to any particular material, however, a thermoplastic resin material may be used such as, for example, polypropylene, polycarbonate, polyamide, polysulfone, polyarylate, methacrylate-butylene-styrene copolymer, and/or the like.

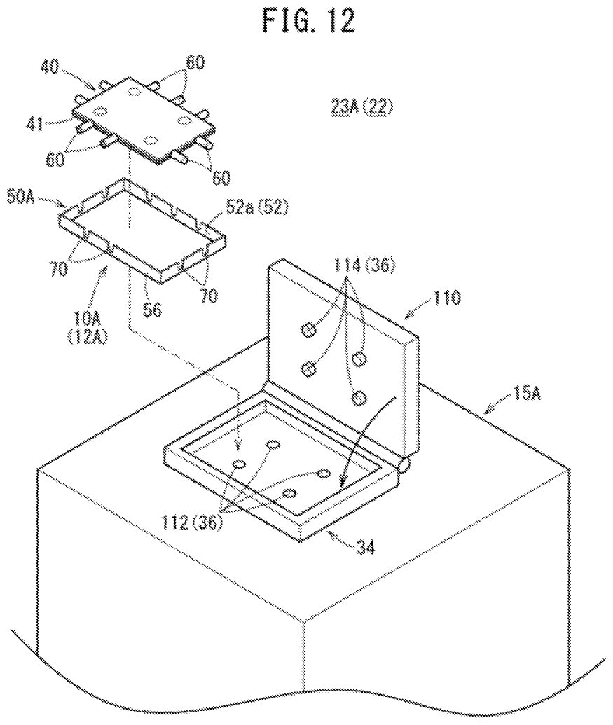

The frame 50 includes a substantially rectangular shaped cover portion 54 which is slightly larger than the cassette main body 40, and side portions 56 that protrude a distance from the outer periphery of the cover portion 54 in a direction perpendicular to the cover portion 54. The side portions 56 may extend around the entire outer periphery or part of the periphery of the cover portion 54. In the frame 50, the accommodation chamber 52 is opened through an opening 52a surrounded by the side portions 56 on an opposite side of the cover portion 54, thereby causing one surface (e.g., the surface visible in FIG. 2) of the cassette main body 40 to be exposed to the external environment. Stated another way, the accommodation chamber 52 is defined by a concave shape that is formed by the side portions 56 and a bottom portion 52b of the accommodation chamber 52, and the other surface of the cassette main body 40 is covered by the bottom portion 52b of the accommodation chamber 52. In the accommodation chamber 52, the bottom portion 52b may include the cover portion 54, and as will be described later, the bottom portion 52b may be open without the presence of the cover portion 54 (see, e.g., FIG. 12).

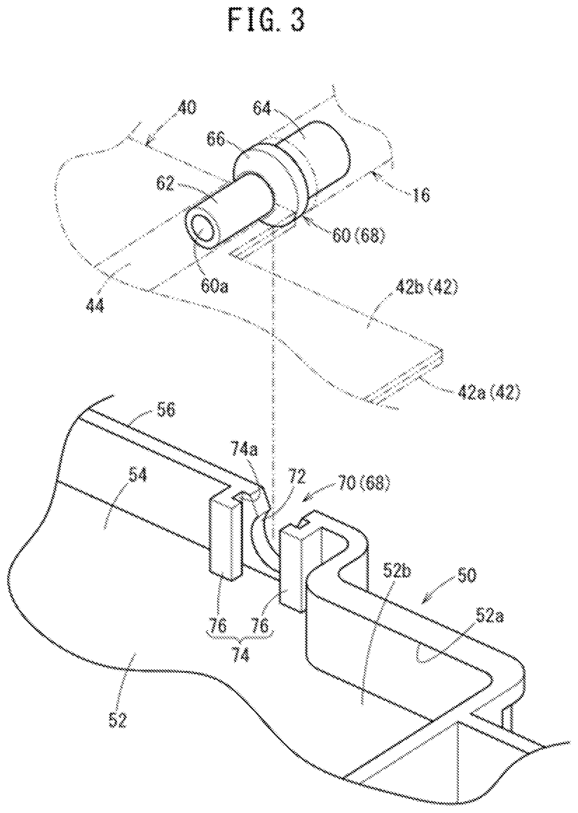

As shown in FIGS. 2 and 3, engaging portions 70 in which the respective connectors 60 are arranged and retained are provided in the side portions 56 at positions corresponding to each of the connectors 60 of the cassette main body 40. The connectors 60 and the engaging portions 70 form engagement mechanisms 68 for engagement with the cassette main body 40.

As shown in FIG. 3, each of the connectors 60 of the cassette main body 40 includes a first cylindrical part 62 that is sealed to the cassette main body 40, a second cylindrical part 64 connected to the tube 16, and a flange 66 that protrudes radially outward between the first cylindrical part 62 and the second cylindrical part 64. Further, a communication hole 60a that penetrates through the first cylindrical part 62, the second cylindrical part 64, and the flange 66 is formed in the axial center of the connector 60 to allow the flow of fluid through the connector 60.

At a time of sealing, when the two resin sheets 42 of the cassette main body 40 are placed together and sealed, the first cylindrical parts 62 may be fusion bonded to the cassette main body 40 so that the communication holes 60a fluidly communicate with the flow paths 44 of the cassette 10. An outer circumferential surface of the first cylindrical part 62 is formed with a smaller diameter than the second cylindrical part 64 in order to correspond with a flow path 44 of the cassette 10. Further, the second cylindrical part 64 are inserted inside a tube 16, and is firmly fixed to the tube 16 by an appropriate fixing method (e.g., adhesive, form-fit, and/or the like). The flanges 66 have a thickness (e.g., a predetermined thickness) in the axial direction of the connectors 60, and may be formed in a ring shape that encircles the entire outer circumferential surface of the connectors 60.

On the other hand, the engaging portions 70 of the frame 50 include engagement recesses 72 in which the side portions 56 are cut out, and movement limiters 74 that protrude from the side portions 56 toward the inner side of the frame 50 in close proximity to the engagement recesses 72. The engagement recesses 72 are opened in the same direction as the opening 52a of the frame 50, and are formed in arcuate shapes (C-shapes) which are capable of accommodating the tubes 16 connected to the connectors 60 (second cylindrical parts 64). The engagement recesses 72 are set to a size that enables them to be firmly fitted with respect to the accommodated tubes 16 and the connectors 60.

Each of the movement limiters 74 may comprise a slot-like structure that accommodate a corresponding flange 66. For example, each movement limiter 74 may include a pair of hook portions 76 which protrude inwardly from the inner surface of the side portions 56, and are bent in perpendicular directions and directions to approach mutually toward each other. The movement limiters 74 allow the flanges 66 of the connectors 60 to be accommodated in fixed spaces 74a formed between the movement limiters 74 and the side portions 56.

Movement of the connectors 60 in the axial direction is restricted by disposing the flanges 66 in the fixed spaces 74a. Further, the connectors 60 are accommodated in the engagement recesses 72 together with the tubes 16 so that the connectors 60 become engaged with the engaging portions 70 (side portions 56) at an appropriate engagement force to prevent the connectors 60 from slipping out from the frame 50. By the respective connectors 60 of the cassette main body 40 being retained by the respective engaging portions 70 of the frame 50, the cassette 10 becomes placed in a state in which the cassette main body 40 and the frame 50 are integrated (placed in a state in which they can be handled together collectively).

Returning to FIG. 2, the aforementioned engagement mechanisms 68 are disposed respectively on four sides (or edges) of the substantially rectangular shaped cassette 10. More specifically, the cassette main body 40 is equipped with the connectors 60 at each of four outer edges 41, whereas the frame 50 is also equipped with the engaging portions 70 at each of four side portions 56. Consequently, the frame 50 is capable of retaining the sheet-like shaped cassette main body 40 in a stretched state, and can suitably cause the flow paths 44 to be extended along a planar direction.

In addition, the cassette 10 is attached to the cell cleaning device 15 in a state in which the cassette main body 40 and the frame 50 are integrated, and with the surface opposite to the illustrated surface in FIG. 2 being held in a reverse manner. Accordingly, in a mounted state, one surface of the cassette main body 40 looking out from the opening 52a of the frame 50 faces toward an upper surface of the cell cleaning device 15, and the cover portion 54 covers the entirety of the cassette main body 40. Stated otherwise, in the mounted state, the upper and lower sides of the cassette main body 40 in FIG. 2 are inverted.

Next, with reference to FIG. 4, a description will be given in detail concerning the paths for the liquids included in the kit 12, together with the flow paths 44 provided in the cassette 10. The cassette 10 of the illustrated example is drawn in a plan view in a state in which the cassette 10 is attached to the cell cleaning device 15. In order to facilitate description, FIG. 4 omits the cover portion 54 of the frame 50. The outer edges 41 of the cassette main body 40 in the illustrated example include a first short (or first transverse) side 41a (left side in the figure), a second short (or second transverse) side 41b (right side in the figure), a first long (or first longitudinal) side 41c (upper side in the figure), and a second long (or second longitudinal) side 41d (lower side in the figure).

The culture tube 16A that is joined to the culture bag 18A is connected to the first long side 41c. The first cleaning solution tube 16B1 that is joined to the first cleaning solution bag 18B1, the second cleaning solution tube 16B2 that is joined to the second cleaning solution bag 18B2, and the product tube 16C that is joined to the product bag 18C are connected to the second short side 41b. The first and second treatment tubes 16D1 and 16D2 that are joined to the treatment unit 20 are connected to the second long side 41d.

Further, in the cell cleaning system 23, in a state in which the cell cleaning device 15 and the kit 12 are set, the three pumps 24 are arranged at positions in proximity to the sides of the cassette 10. More specifically, in the set state, the cassette setting location 34 includes a first pump 24a disposed in proximity to the first short side 41a, a second pump 24b disposed in proximity to the first long side 41c, and a third pump 24c disposed in proximity to the second long side 41d. Therefore, the respective pump tubes 16E of the kit 12 may include a first pump tube 16E1 connected to the first short side 41a, a second pump tube 16E2 connected to the first long side 41c, and a third pump tube 16E3 connected to the second long side 41d.

The first to third pump tubes 16E1 to 16E3 are arranged in a manner so that the portions that are folded back in an arcuate shape are wrapped around circular shaped wound portions of the first to third pumps 24a to 24c. For example, by being vigorously rotated around the respective wrapped around pump tubes 16E, the first to third pumps 24a to 24c apply a fluid force to the liquids in each of the pump tubes 16E.

Furthermore, in the cell cleaning system 23, in a state in which the kit 12 is set with respect to the cell cleaning device 15, the plurality of clamps 26 are arranged at positions in proximity to the sides of the cassette 10. The plurality of clamps may be embodied as mechanical clamps and/or as controllable valves. More specifically, in the set state, the cassette setting location 34 includes first and second clamps 26a and 26b disposed in proximity to the first long side 41c, third to sixth clamps 26c to 26f disposed in proximity to the second short side 41b, and a seventh clamp 26g disposed in proximity to the second long side 41d.

The culture tube 16A is arranged on a first clamp 26a, the first cleaning solution tube 16B1 is arranged on a third clamp 26c, the second cleaning solution tube 16B2 is arranged on a fourth clamp 26d, and the product tube 16C is arranged on a sixth clamp 26f. Further, the clamp tubes 16F include a first clamp tube 16F1 connected to the first long side 41c and arranged on the second clamp 26b, a second clamp tube 16F2 connected to the second short side 41b and arranged on the fifth clamp 26e, and a third clamp tube 16F3 connected to the second long side 41d and arranged on the seventh clamp 26g.

In order to reliably carry out opening and closing of the flow paths 44 of the tubes 16 by the clamps 26, the frame 50 of the cassette 10 includes a plurality of retaining frames 58 that extend from the side portions 56 and retain the tubes 16. The respective retaining frames 58 are provided on the side portions 56 corresponding to the first and second long sides 41c and 41d and the second short side 41b, and retain outer peripheries of the tubes 16 that are separated by a distance (e.g., a predetermined distance) from the side portions 56.

The flow paths 44 of the cassette main body 40 fluidly communicate (sometimes referred to herein as “communicate” or the like) with the respective tubes 16 via each of the connectors 60. The flow paths 44 include a first path 44a that places one end of the first pump tube 16E1 in communication with the culture tube 16A, a second path 44b that places the other end of the first pump tube 16E1 in communication with the first treatment tube 16D1, a third path 44c that places a connection point alpha of the first path 44a in communication with one end of the first clamp tube 16F1, a fourth path 44d that places a connection point beta of the second path 44b in communication with one end of the third clamp tube 16F3, a fifth path 44e that places the other end of the third clamp tube 16F3 in communication with the other end of the first clamp tube 16F1, a sixth path 44f that places a connection point gamma of the fifth path 44e in communication with one end of the second pump tube 16E2, a seventh path 44g that places a first flow path switch 46a provided on the second short side 41b in communication with the other end of the second pump tube 16E2, an eighth path 44h that places the first flow path switch 46a in communication with the first cleaning solution tube 16B1, a ninth path 44i that places the first flow path switch 46a in communication with the second cleaning solution tube 16B2, a tenth path 44j that places the first flow path switch 46a in communication with one end of the second clamp tube 16F2, an eleventh path 44k that places the second flow path switch 46b provided on the second short side 41b in communication with the other end of the second clamp tube 16F2, a twelfth path 441 that places the second flow path switch 46b in communication with the product tube 16C, a thirteenth path 44m that places the second flow path switch 46b in communication with the other end of the third pump tube 16E3, and a fourteenth path 44n that places the second treatment tube 16D2 in communication with one end of the third pump tube 16E3.

At the connection points alpha, beta, and gamma, the first flow path switch 46a, and the second flow path switch 46b, the connected flow paths 44 communicate with each other, and the liquid in one of the flow paths 44 is allowed to flow freely to the other of the flow paths 44. It should be noted that, although the first flow path switch 46a has two connection points for connecting the seventh to tenth paths 44g to 44j, to facilitate explanation, the connection points are considered together as a single entity.

Further, the cassette main body 40 includes detection channel portions 48 for detecting the pressure of the flow paths 44. The respective detection channel portions 48 are capable of detecting the pressure of the liquid flowing therein by being gripped by later-described gripper members 90 (supports) that are provided on the frame 50, and by load cells 100 of the cell cleaning device 15. The configuration of the detection channel portions 48 will be described in detail later.

On the other hand, returning to FIG. 1, the cell cleaning device 15 to which the kit 12 is attached includes a box-shaped device main body 80, a rotor 82 rotatably accommodated inside the device main body 80, and a stand 84 on which the respective medical bags 18 of the kit 12 are held. Further, a display 86 by which operations and display are carried out when the cleaning treatment is performed, and the aforementioned cassette setting location 34 are provided on outer surfaces of the device main body 80. Furthermore, on the device main body 80, a control unit or controller 88 is provided for controlling operations of the cell cleaning system 23.

The rotor 82 may have a cylindrical shape, is disposed below the cassette setting location 34, and is rotated about an axis by a non-illustrated rotary drive source that is provided in the device main body 80. By undergoing rotation in a state with the treatment unit 20 (treatment case 28) of the kit 12 being accommodated therein, the rotor 82 applies a centrifugal force to the liquid that flows into the treatment unit 20.

Further, the cassette setting location 34 exhibits a frame structure, which is formed on the upper inclined surface of the device main body 80, and is formed in a manner so that, when the cassette 10 is fitted on the inner side of the frame structure, the frame 50 is locked by non-illustrated hooks.

As described above, the cassette setting location 34 is equipped with the first to third pumps 24a to 24c and the first to seventh clamps 26a to 26g on the outer peripheral side of the frame structure and may enable placement of the retaining frames 58 of the cassette 10. Upon the attachment of the cassette 10 to the cassette setting location 34 (e.g., by the user) the plurality of pumps 24 and the clamps 26 are arranged in relation to appropriate tubes 16 of the kit 12.

Further, the cell cleaning system 23 according to the present embodiment detects the pressure (state) of the liquid that flows in predetermined flow paths 44 inside the cassette 10. More specifically, the cell cleaning system 23 comprises pressure detectors 36 in each of the first path 44a, the second path 44b, the sixth path 44f, the seventh path 44g, the thirteenth path 44m, and the fourteenth path 44n inside the cassette 10. Stated otherwise, each of the pressure detectors 36 is provided respectively on an upstream side and a downstream side of the first to third pumps 24a to 24c. In addition, on the basis of the pressures (e.g., differential pressures) of the respective pressure detectors 36, the control unit 88 of the cell cleaning device 15 calculates the flow rates at times when the first to third pumps 24a to 24c are driven. The calculated flow rates are fed back to assist with the control of the first to third pumps 24a to 24c.

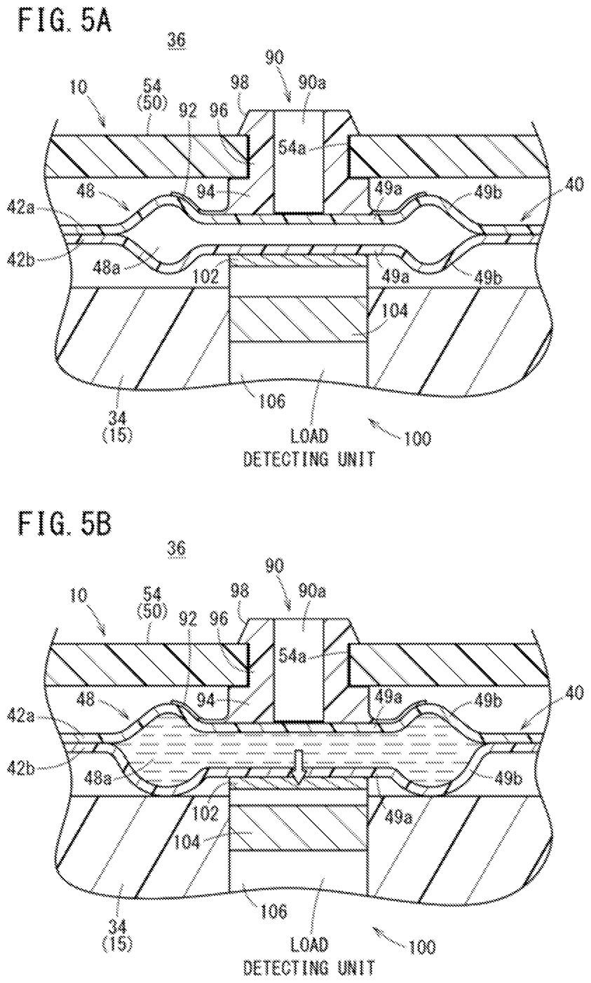

As shown in FIG. 5A, in a set state of the cassette 10 and the cell cleaning device 15, each of the pressure detectors 36 may include a detection channel portion 48 and a gripper member 90 of the cassette 10, and a load cell 100 of the cell cleaning device 15. The respective pressure detectors 36 sandwich (e.g., grip) the detection channel portions 48 by the gripper members 90 and the load cells 100, and detect the loads (e.g., pressures) of the detection channel portions 48 when the liquid flows through the detection channel portions 48.

In a plan view of the cassette main body 40, the detection channel portions 48 extend in a planar direction and in a circular shape (e.g., a substantially perfect circular shape with respect to the contiguously disposed flow paths 44. The detection channel portions 48 include flat portions 49a having circular shapes (e.g., substantially perfect circular shapes) formed on each of the two resin sheets 42, and protruding portions 49b connected to the circumferential edge of the flat portions 49a and protruding from the flat portions 49a. Between the pairs of flat portions 49a and the pairs of protruding portions 49b, flow chambers 48a are formed through which the liquid flows in the detection channel portions 48.

In addition, the gripper members 90 are attached beforehand to the flat portions 49a of the resin sheet 42a on the side of the frame 50 (e.g., cover portion 54). On the other hand, in the set state, the load cells 100 are disposed on the flat portions 49a of the resin sheet 42b on the side of the cell cleaning device 15 (e.g., at cassette setting location 34).

The gripper members 90 are configured to be harder (e.g., less flexible) than the cassette main body 40, and are fixed to the detection channel portions 48 by an appropriate fixing method (e.g., an adhesive). The gripper members 90 may be attached to holes 54a disposed at positions opposing the detection channel portions 48 of the frame 50 (e.g., cover portion 54). The gripper members 90, by being fixed to the cover portion 54, fix or support the resin sheet 42a on the side of the frame 50 of the cassette main body 40 (at the detection channel portions 48). The material constituting the gripper members 90 is not particularly limited, and for example, may include the resin material cited for the frame 50, or a metal material.

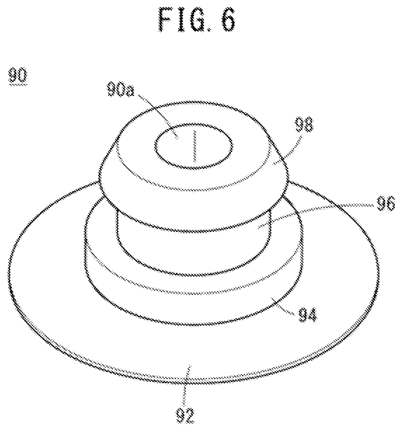

As shown in FIG. 6, each of the gripper members 90 is formed in a cylindrical shape having an axis along a thickness direction of the cover portion 54 of the frame 50, and is formed with convex and concave portions on the outer circumferential surface thereof. More specifically, each of the gripper members 90 includes a portion to be fixed 92 (e.g., that is fixed to the resin sheet 42a), a first coming-off prevention part 94, a body portion 96, and a second coming-off prevention part 98, arranged in this order from the one end, which is fixed to the detection channel portion 48, to the other end.

The portions to be fixed 92 are portions that may be directly fixed to the detection channel portions 48, and may possess flexibility as a result of being formed in the gripper members 90 to have a largest diameter and a thinnest disk-like shape. Concerning the central part of the portions to be fixed 92, the flatness thereof is maintained by the first coming-off prevention parts 94. On the other hand, the outer circumferential part of the portions to be fixed 92 can be easily deformed as show in FIGS. 5A and 5B.

In addition, in the portions to be fixed 92, the central parts are fixed to the flat portions 49a of the detection channel portions 48, and the outer parts are fixed to the protruding portions 49b, thereby being appropriately deformed to correspond to the shapes of the flat portions 49a and the protruding portions 49b, and the entire end surfaces thereof are fixed to the resin sheet 42a. The fixing means for fixing the detection channel portions 48 to the gripper members 90 is not particularly limited, and for example, fusion bonding or adhesion may be used. Moreover, the detection channel portions 48 and the gripper members 90 may be configured to contact one another without being fixed to each other.

Although formed to be smaller in diameter than the portions to be fixed 92, the first coming-off prevention parts 94 are formed in an annular shape that is larger in diameter than the holes 54a of the frame 50. The first coming-off prevention parts 94 prevent the gripper members 90 from coming off toward the opposite side of the cassette main body 40, and serve to maintain a constant distance from the cover portion 54 to the detection channel portions 48.

The body portions 96 are thinner than the first coming-off prevention parts 94, and are formed to have a diameter substantially equivalent to the holes 54a of the frame 50 (so as to be capable of fitting into the holes 54a). The body portions 96 are formed to have the same length as the thickness of the cover portion 54 of the frame 50. Consequently, the first and second coming-off prevention parts 94 and 98 come into surface contact with the cover portion 54 in a state in which the body portions 96 are disposed in the holes 54a.

The second coming-off prevention parts 98 are formed in an annular shape with a diameter larger than that of the holes 54a of the frame 50 (roughly the same as the diameter of the first coming-off prevention parts 94). The second coming-off prevention parts 98 prevent the gripper members 90 from coming off toward the side of the cassette main body 40. By the gripper members 90 including the holes 90a in the first coming-off prevention parts 94, the body portions 96, and the second coming-off prevention parts 98, elastic deformation of the second coming-off prevention parts 98 is facilitated. Further, the outer circumferential surface of the second coming-off prevention parts 98 is formed in a tapered shape. Consequently, attachment of the gripper members 90 with respect to the frame 50 can be smoothly performed.

Upon formation of the cassette 10 (e.g., when the cassette main body 40 is manufactured), the cassette main body 40 and the gripper members 90 may be integrated by fixing the gripper members 90 to the resin sheets 42. In addition, when the cassette main body 40 is attached to the frame 50, the second coming-off prevention parts 98 of the gripper members 90 are inserted into the holes 54a of the cover portion 54, and the cover portion 54 is arranged between the first and second coming-off prevention parts 94 and 98.

Consequently, in the cassette 10, the positions of the detection channel portions 48 and the gripper members 90 can be accurately set. Further, since the cassette main body 40 is retained by the frame 50 at a location other than where the connectors 60 are retained with respect to the frame 50, the planar state thereof is stably maintained. Moreover, the configuration for supporting the detection channel portions 48 of the cassette main body 40 is not particularly limited, and for example, the gripper members 90 may be formed integrally with the frame 50.

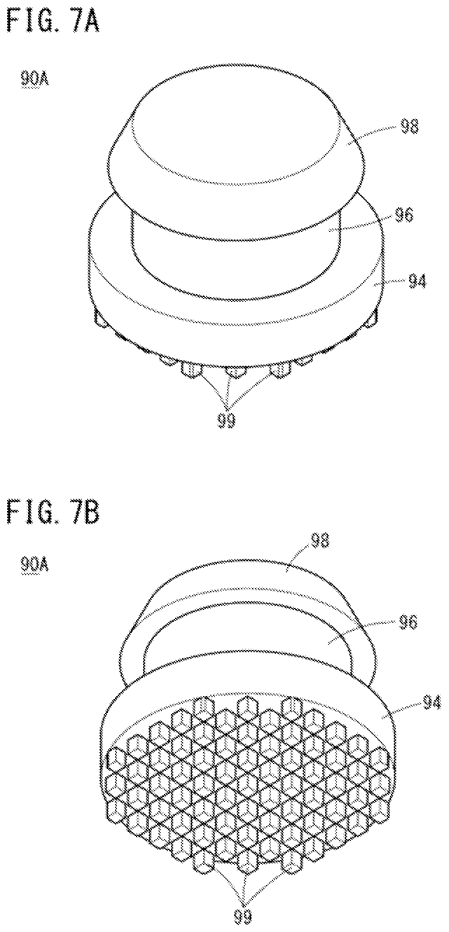

Further, in the cassette 10 (kit 12), the gripper members 90A according to a modification shown in FIGS. 7A and 7B may be applied. Instead of the portions to be fixed 92 described above, the gripper members 90A include a plurality of protrusions 99 at end portions on the side of the cassette main body 40. The plurality of protrusions 99 are fixed to the detection channel portions 48 (flat portions 49a) of the one resin sheet 42a, thereby restricting displacement of the flat portions 49a. Further, in the plurality of protrusions 99, an adhesive or a molten material or the like can be made to enter into gaps between the protrusions 99, thereby causing the height position of the resin sheet 42 held by the gripper members 90 to be kept constant more accurately.

On the other hand, concerning the load cells 100 of the cell cleaning device 15, for example, a load measurement method is applied thereto. The load cells 100 may comprise any suitable type of load cell for sensing fluid pressure in the context of a biological component treatment system. Examples of a load cell 100 include a hydraulic load cell, a strain gauge load cell, a capacitive load cell, a pneumatic load cell, and/or magnetic load cell. In one example, each of the load cells 100 is equipped with a displaceable member 102 (e.g., comprising a metal, such as steel) provided at the cassette setting location 34, a fixing magnet 104 disposed at a position in proximity to a lower side of the displaceable 102, and a load detector 106 having the fixing magnet 104 mounted thereon.

The displaceable members 102 are formed in a shape (e.g., circular shape) that coincides substantially with the shape of the flat portions 49a, and in the case that liquid is not flowing through the detection channel portions 48 and almost no load is received, the displaceable members 102 cooperate with the gripper members 90 to make the interval between the pairs of flat portions 49a substantially constant. In addition, the displaceable members 102 are displaced when loads are received from the detection channel portions 48 of the cassette main body 40, and approach toward the fixing magnets 104.

Accordingly, the load detectors 106 detect changes in the loads of the fixing magnets 104 in accordance with the displacement of the displaceable members 102, and transmit load detection signals of the detection channel portions 48 to the control unit 88 of the cell cleaning device 15.

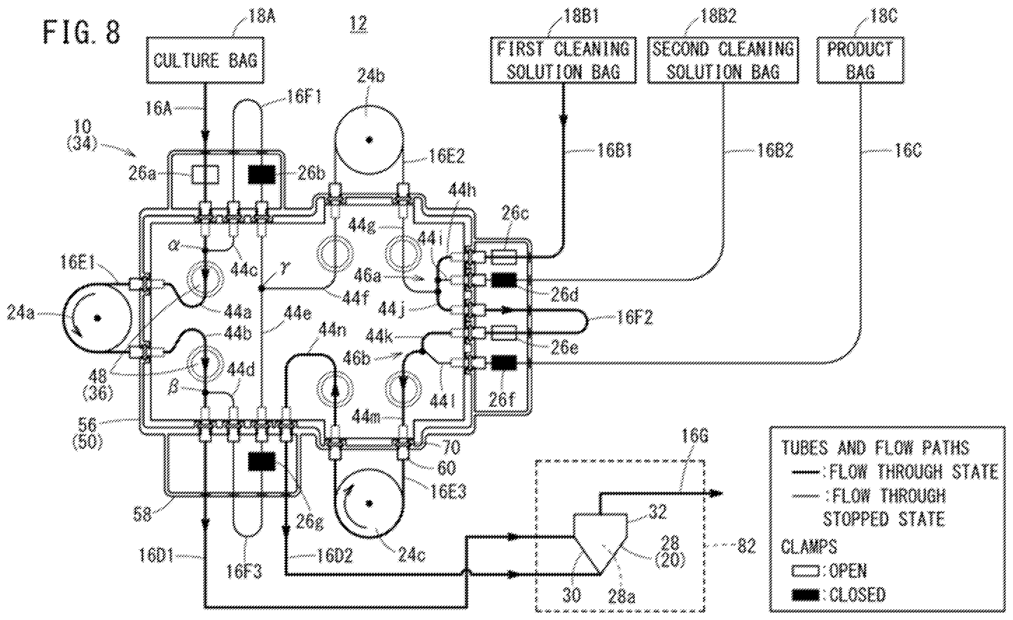

Next, with reference to FIGS. 8 to 11, a description will be given concerning operations of the tubes 16, and operations of the flow paths 44 of the cassette 10 in a cleaning treatment of the cell cleaning system 23. The various elements shown in FIGS. 8 to 11 may be controlled by the control unit 88. Although not explicitly shown, it should be appreciated that the control unit 88 may include one or more processing circuits for carrying out tasks in the system 23, for example, tasks associated with processing blood and/or controlling the flow of fluid. Such processing circuits may be included in the cell cleaning device 15 and may comprise software, hardware, or a combination thereof. For example, a processing circuit may include a memory including executable instructions and at least one processor (e.g., a microprocessor) that executes the instructions on the memory. The memory may correspond to any suitable type of memory device or collection of memory devices configured to store instructions. Non-limiting examples of suitable memory devices that may be used include Flash memory, Random Access Memory (RAM), Read Only Memory (ROM), variants thereof, combinations thereof, or the like. In some embodiments, the memory and processor may be integrated into a common device (e.g., a microprocessor may include integrated memory). Additionally or alternatively, a processing circuit may comprise hardware, such as an application specific integrated circuit (ASIC). Other non-limiting examples of the processing circuits include an Integrated Circuit (IC) chip, a Central Processing Unit (CPU), a microprocessor, a Field Programmable Gate Array (FPGA), a collection of logic gates or transistors, resistors, capacitors, inductors, diodes, or the like. Some or all of the processing circuits may be provided on a Printed Circuit Board (PCB) or collection of PCBs. It should be appreciated that any appropriate type of electrical component or collection of electrical components may be suitable for inclusion in the processing circuits.

In the cleaning treatment of the cell cleaning system 23, the operator sets the kit 12 including the cassette 10 in the cell cleaning device 15, and as shown in FIG. 4, arranges or sets the kit 12 with respect to the pumps 24 and the clamps 26 of the cell cleaning device 15. After such setting is completed, the cleaning treatment begins and a priming step is executed. In the priming step, the cell cleaning device 15 appropriately operates the first to third pumps 24a to 24c, and supplies the cleaning solution of the first or second cleaning solution bag 18B1, 18B2 into the flow paths 44 of the cassette 10, and into the internal space 28a of the treatment case 28. Consequently, the air in the flow paths 44 and the internal space 28a escapes, and the flow paths 44 and the internal space 28a are filled with the cleaning solution.

After completion of the priming step, as shown in FIG. 8, in the cleaning treatment, a culture supplying step for the first time (first culture supplying step) for supplying the culture in the culture bag 18A to the treatment case 28 is performed. In the culture supplying step, the cell cleaning device 15 drives the first pump 24a, and transfers the culture to the treatment unit 20 by placing in communication a path from the culture bag 18A to the treatment unit 20. Therefore, the cell cleaning device 15 opens the first clamp 26a, together with closing the second clamp 26b and the seventh clamp 26g. Consequently, the culture in the culture bag 18A passes through the culture tube 16A, the first path 44a, the first pump tube 16E1, and the second path 44b, and flows to the first treatment tube 16D1, whereupon the culture flows into the internal space 28a from the side of the treatment case 28. At the time that the culture is transferred, the cell cleaning device 15 rotates the rotor 82 at an appropriate speed of rotation (for example, 3000 rpm), and applies a centrifugal force to the treatment case 28.

Furthermore, in the cell cleaning system 23 according to the present embodiment, in a culture supplying step, the cleaning solution is supplied to the treatment case 28, in conjunction with the culture being supplied to the treatment case 28. Therefore, in the cell cleaning device 15, the third clamp 26c and the fifth clamp 26e are opened, while on the other hand, the fourth clamp 26d and the sixth clamp 26f are closed, and furthermore, the third pump 24c is driven.

Consequently, the cleaning solution in the first cleaning solution bag 18B1 passes through the first cleaning solution tube 16B1, the eighth path 44h, the tenth path 44j, the second clamp tube 16F2, the eleventh path 44k, the thirteenth path 44m, the third pump tube 16E3, and the fourteenth path 44n, and flows to the second treatment tube 16D2, whereupon the cleaning solution flows into the internal space 28a from the apex of the treatment case 28.

In the culture supplying step, the amount of the culture supplied to the treatment case 28 is set, for example, at a value from 5 mL/min to 50 mL/min, and the amount of the cleaning solution supplied to the treatment case 28 is set, for example, to 2.5 mL/min. Consequently, in the cell cleaning system 23, by preventing the cells of the culture inside the treatment case 28 from being strongly pressed outward in the centrifugal direction by the cleaning solution, the cells can be protected. Since the specific gravity of the cleaning solution and the culture medium contained within the culture inside the treatment case 28 is lower than that of the cells, the cleaning solution and the culture medium are moved to the bottom of the treatment case 28 by the centrifugal force, and flow out to the disposal tube 16G.

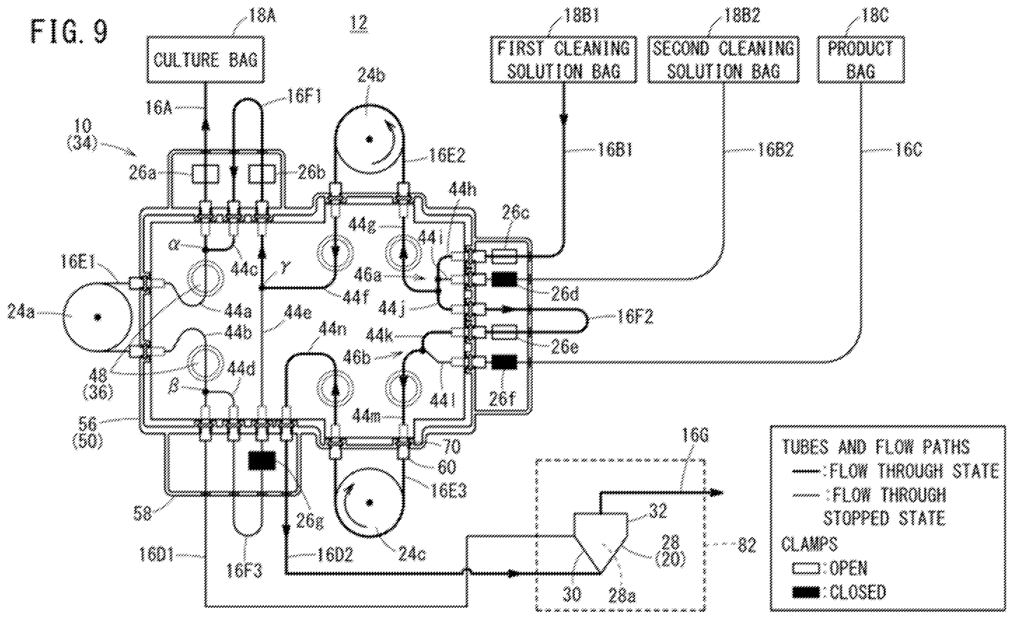

Next, in the cleaning treatment, as shown in FIG. 9, the cleaning solution is supplied to the culture bag 18A, and a peeling step is carried out to peel off or remove cells that are adhered to the culture bag 18A. In the peeling step, the cell cleaning device 15 places in communication a path from the first cleaning solution bag 18B1 to the culture bag 18A. More specifically, the cell cleaning device 15 opens the first clamp 26a, the second clamp 26b, and the third clamp 26c, together with closing the fourth clamp 26d and the seventh clamp 26g. Consequently, the cleaning solution in the first cleaning solution bag 18B1 passes through the first cleaning solution tube 16B1, the eighth path 44h, the seventh path 44g, the second pump tube 16E2, the sixth path 44f, the fifth path 44e, the first clamp tube 16F1, the third path 44c, and the first path 44a, and flows to the culture tube 16A, whereupon the cleaning solution flows into the culture bag 18A.

Moreover, at the time of the peeling step, the cleaning solution may be supplied to the treatment case 28, and further, the rotor 82 may be rotated to apply a centrifugal force to the treatment case 28. More specifically, in the cell cleaning device 15, the third clamp 26c and the fifth clamp 26e are opened, while on the other hand, the fourth clamp 26d and the sixth clamp 26f are closed, and furthermore, the third pump 24c is driven. Consequently, inside the treatment case 28, centrifugation of the culture, and disposal of the culture medium and the cleaning solution continue to progress.

In addition, in the cell cleaning system 23, after completion of the peeling step, a culture supplying step for the second time (second culture supplying step) is performed. In the cell cleaning device 15, by performing the same operations as in the first culture supplying step in the second culture supplying step as well, the cells that were peeled off in the peeling step are supplied to the treatment case 28 (see FIG. 8). Additionally, in the cell cleaning device 15, the peeled off culture in the treatment case 28 is subjected to centrifugation, and further, the culture medium is discarded from the treatment case 28 together with the supplied cleaning solution. Consequently, the process can be performed without leaving cells inside the culture bag 18A.

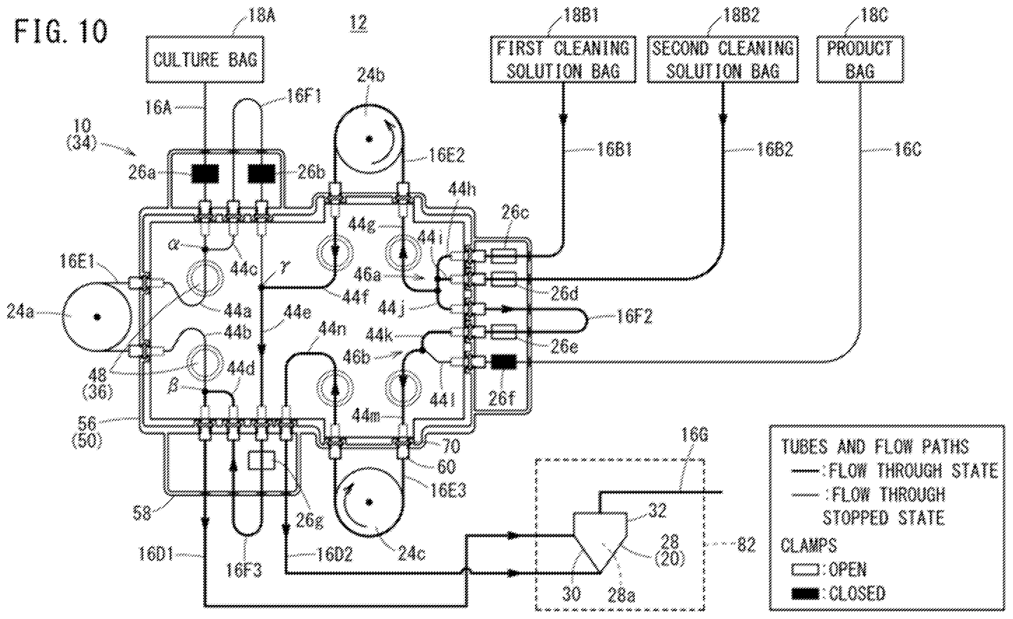

After completion of the second culture supplying step, in the cell cleaning system 23, as shown in FIG. 10, a cleaning step of further supplying the cleaning solution to the treatment case 28 to thereby clean the cells (remove the medium) is carried out. In the cleaning step, the cell cleaning device 15 places in communication a path from the first and second cleaning solution bags 18B1, 18B2 to the treatment case 28. More specifically, the third clamp 26c, the fourth clamp 26d, the fifth clamp 26e, and the seventh clamp 26g are opened, while on the other hand, the first clamp 26a, the second clamp 26b, and the sixth clamp 26f are closed. Further, by the cell cleaning device 15 driving the second and third pumps 24b and 24c, the cleaning solution of the first and second cleaning solution bags 18B1 and 18B2 passes through two paths (the first and second treatment tubes 16D1 and 16D2), and is supplied to the treatment case 28. Furthermore, in the cleaning step as well, the cell cleaning device 15 causes the rotor 82 to rotate, and applies a centrifugal force to the treatment case 28.

In this case, the cleaning solution in the first and second cleaning solution bags 18B1 and 18B2 flows into the flow paths 44 (the eighth path 44h and the ninth path 44i) of the cassette 10 through each of the first and second cleaning solution tubes 16B1 and 16B2, and flows through the first flow path switch 46a. In addition, as one of the paths, the cleaning solution passes through the seventh path 44g, the second pump tube 16E2, the sixth path 44f, the fifth path 44e, the third clamp tube 16F3, the fourth path 44d, and the second path 44b, and then flows into the first treatment tube 16D1, whereupon the cleaning solution flows into the internal space 28a from the side of the treatment case 28. Further, as another of the paths, the cleaning solution passes through the tenth path 44j, the second clamp tube 16F2, the eleventh path 44k, the thirteenth path 44m, the third pump tube 16E3, and the fourteenth path 44n, and flows to the second treatment tube 16D2, whereupon the cleaning solution flows into the internal space 28a from the apex of the treatment case 28. Consequently, inside the treatment case 28, removal of the medium that was subjected to centrifugation further progresses is increased, and the cell concentration is increased.

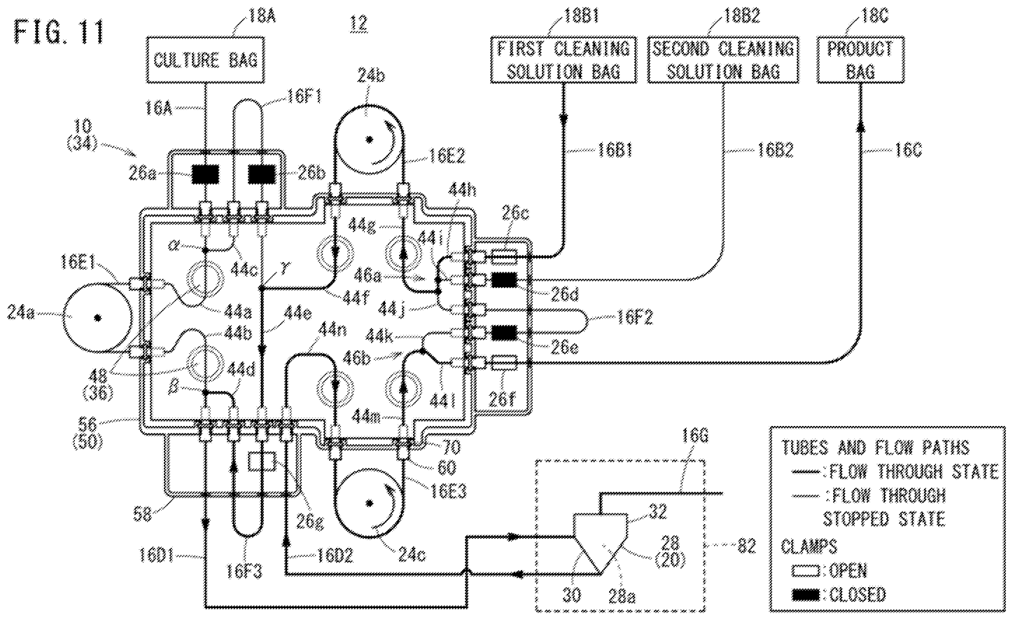

After completion of the cleaning step, upon treatment of the cells, as shown in FIG. 11, a product transfer step is executed to transfer the cleaned cells inside the treatment case 28 into the product bag 18C. In the product transfer step, the cell cleaning device 15 places in communication a path from the treatment case 28 to the product bag 18C, and places in communication a path from the first cleaning solution bag 18B1 to the treatment case 28. For this purpose, in the cell cleaning device 15, the third clamp 26c, the sixth clamp 26f, and the seventh clamp 26g are opened, while on the other hand, the first clamp 26a, the second clamp 26b, the fourth clamp 26d, and the fifth clamp 26e are closed. Further, the cell cleaning device 15 drives the second and third pumps 24b and 24c, together with causing the rotor 82 to rotate at a lower speed of rotation (for example, 2400 rpm) than in the cleaning step or the like.

Consequently, the cleaning solution in the first cleaning solution bag 18B1 passes through the first cleaning solution tube 16B1, the eighth path 44h, the seventh path 44g, the second pump tube 16E2, the sixth path 44f, the fifth path 44e, the third clamp tube 16F3, the fourth path 44d, and the second path 44b, and flows into the first treatment tube 16D1, whereupon the cleaning solution flows into the internal space 28a from the side of the treatment case 28. Further, the cells inside the treatment case 28 (including the cleaning solution) pass through the second treatment tube 16D2, the fourteenth path 44n, the third pump tube 16E3, the thirteenth path 44m, and the twelfth path 441, and flow to the product tube 16C, whereupon the cells flow into the product bag 18C from the product tube 16C. In the product transfer step, the flow rate of the cleaning solution by the second pump 24b, and the flow rate of the product (cells) by the third pump 24c are set at the same rate (for example, 40 mL/min).

By the above process, the cleaned cells (e.g., highly concentrated cells) are stored in the product bag 18C, whereupon the cleaning treatment of the cell cleaning system 23 is brought to an end. After completion of the cleaning treatment, the operator cuts and seals the product tube 16C of the kit 12, and separates the product bag 18C from the kit 12. The kit 12 including the cassette 10 is removed from the cell cleaning device 15 and is discarded.

In the aforementioned cleaning treatment, the pressure is detected in each of the pressure detectors 36 of the respective flow paths 44 (the first path 44a, the second path 44b, the sixth path 44f, the seventh path 44g, the thirteenth path 44m, and the fourteenth path 44n) of the cell cleaning system 23. As shown in FIGS. 5A and 5B, in the pressure detectors 36, the gripper members 90 position the flat portions 49a (detection channel portions 48) of the one resin sheet 42a, whereas the flat portions 49a of the other resin sheet 42b are opposed to the load cells 100.

Accordingly, in the case that liquid is not flowing in the flow chambers 48a of the detection channel portions 48, a pressure is not applied to the flat portions 49a from the interiors of the flow chambers 48a, and the displaceable members 102 of the load cells 100 undergo almost no displacement. Therefore, the load detectors 106 detect a weak pressure (or no pressure). On the other hand, in the case that liquid is flowing in the flow chambers 48a of the detection channel portions 48, a pressure is applied to the flat portions 49a from the liquid of the flow chambers 48a, whereby the displaceable members 102 of the load cells 100 are displaced in a direction to approach the fixing magnets 104. Consequently, the load detection units 106 detect the loads of the fixing magnets 104 in accordance with the displacement of the displaceable members 102, and transmit the same to the control unit 88 of the cell cleaning device 15.

In particular, even if liquid flows in the flow chambers 48a, since the resin sheet 42a on the side of the cover portion 54 is supported by the gripper members 90, the flat portions 49a on the one side of the detection channel portions 48 are not displaced. As a result, the flat portions 49a on the other side may be reliably displaced, whereby the detection of pressure by the load cells 100 can be suitably performed.

The present disclosure is not limited to the above-described embodiment, and various modifications can be adopted in accordance with the essence and gist of the present disclosure. For example, it goes without saying that the shapes of the respective flow paths 44 of the cassette 10 (cassette main body 40) may be freely designed.

As shown in FIG. 12, a cassette 10A (kit 12A, cell cleaning system 23A) according to another embodiment comprises the side portions 56 that surround the cassette main body 40, but differs from the cassette 10 according to the first embodiment in that a frame 50A having an opening 52b without being equipped with the cover portion 54 is applied thereto. In this case, the cell cleaning device 15A includes the cassette setting location 34 in which the cassette 10A is disposed, and a lid 110 for closing the cassette 10A that is attached to the cassette setting location 34.

In such a manner, even if the frame 50 includes only by the side portions 56, the plurality of connectors 60 that protrude from the outer edges 41 of the cassette main body 40 can be supported by the engaging portions 70 of the frame 50 (side portions 56). Thus, the cassette 10A is retained in the frame 50 in a state with the cassette main body 40 being stretched, and is easily set in the cell cleaning device 15.

Further, the pressure detectors 36 of the cell cleaning system 23A may include detectors 112 provided in the cassette setting location 34, and detecting magnets 114 that are provided on the lid 110. More specifically, by the lid 110 being closed in the set state, the detecting magnets 114 come into contact with the detection channel portions 48 on the opposite side from the detectors 112, and the detection channel portions 48 are gripped between the detecting magnets 114 and the detectors 112. Then, when the detecting magnets 114 undergo displacement accompanying the flow of the liquid to the detection channel portions 48, the detectors 112 detect the change in magnetism brought about by the displacement of the detecting magnets 114. Consequently, the detectors 112 can easily detect the pressure of the detection channel portions 48 in the same or similar manner as load detectors 36.

Further, for example, the biological component treated by the biological component treatment system 22 is not limited to cells, and may be extracted components such as blood cell components, lymphocyte components, and the like.

Accordingly, the cassette 10A, the kit 12A, and the biological component treatment device 14 may be included as part of a blood component separation and transfer system. In this case, the kit 12A is configured in the form of a blood kit having a plurality of medical bags 18 and which is capable of distributing whole blood or blood components, and the biological component treatment device 14 is configured in the form of a centrifugal separation and transfer device for centrifuging the blood and transferring the components thereof into appropriate medical bags 18. Further, the cassette 10A is provided in the kit 12A in order to facilitate setting of the kit 12A in the centrifugal separation and transfer device.

Technical concepts and effects that can be grasped from the above-described embodiments will be described below.

The first aspect of the present disclosure is characterized by the biological component cassette 10, 10A that allows the liquid containing the biological component to flow, the biological component cassette 10, 10A comprising the cassette main body 40 having the flow paths 44 for the liquid in the interior of the cassette main body 40, and which is formed into a flexible sheet-like shape, and the frame 50, 50A which is made less flexible than the cassette main body 40 and retains the cassette main body 40, wherein the frame 50, 50A includes the accommodation chamber 52 in which the cassette main body 40 is accommodated, and the side portions 56 forming the accommodation chamber 52, and one surface of the cassette main body 40 is covered by the bottom portion 52b of the accommodation chamber 52, and another surface of the cassette main body 40 is exposed from the frame 50, 50A.

In accordance with the above-described features, in the biological component cassette 10, 10A, the cassette main body 40 which possesses flexibility is retained by the rigid frame 50, 50A. Consequently, the plurality of paths which are used for treatment of the biological component can be easily set in the biological component treatment device 14, and it is possible to enhance working efficiency. In addition, by the side portions 56 exposing the one surface of the cassette main body 40, and the cassette main body 40 which possesses flexibility constituting the flow paths 44, operations of deforming the flow paths 44 or the like can be easily performed with respect to the cassette main body 40, and the state of the liquid in the flow paths 44 can be suitably detected with high accuracy.

Further, the accommodation chamber 52 is defined by a recessed shape formed by the side portions 56 and the bottom portion 52b. In accordance with this feature, the biological component cassette 10, 10A can suitably retain the cassette main body 40 in the accommodation chamber 52 that is defined by the side portions 56 and the bottom portion 52b.

Further, the frame 50 includes the cover portion 54 that forms the bottom portion 52b of the accommodation chamber 52. In accordance with this feature, when the biological component cassette 10 is attached to the device, the cover portion 54 can cover the cassette main body 40 without allowing the cassette main body 40 to be exposed, and the flow paths 44 inside the cassette main body 40 can be operated in a stable manner.

Further, the cover portion 54 includes the supports (gripper members 90, 90A) that protrude toward the cassette main body 40, and contact the one surface of the cassette main body 40 and support the cassette main body 40. Since the biological component cassette 10 has the supports, it becomes possible to sandwich (grip) the detection channel portions 48 in cooperation with the pressure detectors 36 (load cells 100) of the device, and the pressure of the liquid in the detection channel portions 48 can be suitably detected.

Further, the detection channel portions 48 are disposed at positions (e.g., predetermined positions) of the cassette main body 40 through which the flow paths 44 extend in order to detect the state of the liquid by the device to which the biological component cassette 10, 10A is attached, and the supports (gripper members 90, 90A) are arranged at positions opposed to the detection channel portions 48. By the biological component cassette 10, 10A being equipped with the detection channel portions 48 in the cassette main body 40, the state of the liquid flowing through the flow paths 44 can be recognized with high accuracy.

Further, the detection channel portions 48 protrude in a thickness direction of the cassette main body 40, together with including the flat portions 49a extending parallel to the one surface of the cassette main body 40 from the flow paths 44. In accordance with this feature, the flat portions 49a of the detection channel portions 48 are capable of more appropriately detecting the pressure as a state of the liquid flowing through the flow paths 44.

Further, the supports are the gripper members 90, 90A that are attached to the cover portion 54, and the gripper members 90, 90A possess flexibility and include the portions to be fixed 92 that are fixed to the cassette main body 40. Consequently, the gripper members 90 can be attached to the frame 50 in a state of being fixed to the cassette main body 40, so that manufacturing can be performed more easily. Further, the gripper members 90 that are attached to the frame 50 can accurately detect the pressure by gripping the detection channel portions 48.

Further, the outer edges 41 of the cassette main body 40 include the connectors 60 which are in communication with the flow paths 44, and the side portions 56 include the engaging portions 70 that engage with the connectors 60. In this manner, by providing the connectors 60 on the outer edges 41, the biological component cassette 10, 10A can firmly carry out the connection of the cassette main body 40 and the side portions 56 via the connectors 60.

The engaging portions 70 include the engagement recesses 72 which carry out engagement of the connectors 60 to the side portions 56 by inserting the connectors 60 along with the tubes 16 that are connected to the connectors 60. In accordance with this feature, since the biological component cassette 10, 10A includes the engagement recesses 72, the connectors 60 and the tubes 16 are capable of engaging more securely with the engagement recesses 72.

The connectors 60 comprise the flanges 66 that project radially outward from cylindrical portions connected to the cassette main body 40, and the engaging portions 70 comprise the movement limiters 74 that make the connectors 60 immovable in the axial direction of the connectors 60 by accommodating the flanges 66 therein. In the biological component cassette 10, 10A, by making the connectors 60 immovable in the axial direction by the flanges 66 and the movement limiters 74, the cassette main body 40 can be retained in a state of being stretched in a planar direction with respect to the frame 50, 50A. Consequently, positioning of the cassette main body 40 with respect to an external device can be suitably performed.

Further, the cassette main body 40 is formed in a rectangular shape in a plan view, and the connectors 60 are provided on the outer edges 41 of each of four sides of the cassette main body 40, so that the plurality of connectors 60 are engaged with the plurality of the engaging portions 70 provided on the side portions 56. By the connectors 60 on the four outer edges 41 being supported by the engaging portions 70 of the side portions 56, it becomes possible for the biological component cassette 10, 10A to more stably retain the cassette main body 40. Consequently, the liquid is capable of flowing smoothly in the flow paths 44 of the cassette main body 40.

Further, the resin that constitutes the cassette main body 40 is made up from any one of a vinyl chloride resin, a polyolefin resin, and a polyurethane resin. In accordance with this feature, manufacturing costs for the cassette main body 40 can be reduced, and molding accuracy can be increased.

Further, the second aspect of the present disclosure is characterized by the biological component kit 12, 12A comprising the tubes 16 that allow the liquid containing the biological component to flow, and the biological component cassette 10, 10A to which the tubes 16 are connected, wherein the biological component cassette 10, 10A comprises the cassette main body 40 having the flow paths 44 for the liquid in the interior of the cassette main body 40, and which is formed into a flexible sheet-like shape, and the frame 50, 50A which is made less flexible than the cassette main body 40 and retains the cassette main body 40, wherein the frame 50, 50A includes the accommodation chamber 52 in which the cassette main body 40 is accommodated, and the side portions 56 forming the accommodation chamber 52, and one surface of the cassette main body 40 is covered by the bottom portion 52b of the accommodation chamber 52, and another surface of the cassette main body 40 is exposed from the frame 50, 50A. In accordance with such features, the biological component kit 12, 12A, while enabling the flow paths 44 of the cassette main body 40 to be deformed, makes it possible to set the plurality of paths that are formed by the tubes 16 more easily, and can enhance working efficiency.

Further, the third aspect of the present disclosure is characterized by the biological component treatment system 22 comprising the biological component kit 12, 12A having the tubes 16 that allow the liquid containing the biological component to flow, and the biological component cassette 10, 10A to which the tubes 16 are connected, and the biological component treatment device 14 in which the biological component kit 12, 12A is set, wherein the biological component cassette 10, 10A comprises the cassette main body 40 having the flow paths 44 for the liquid in the interior of the cassette main body 40, and which is formed into a flexible sheet-like shape, and the frame 50, 50A which is made less flexible than the cassette main body 40 and retains the cassette main body 40, wherein the frame 50, 50A includes the accommodation chamber 52 in which the cassette main body 40 is accommodated, and the side portions 56 forming the accommodation chamber 52, and one surface of the cassette main body 40 is covered by the bottom portion 52b of the accommodation chamber 52, and another surface of the cassette main body 40 is exposed from the frame 50, 50A, and the biological component treatment device 14 includes the cassette setting location 34 in which the biological component cassette 10, 10A is set. In accordance with such features, the biological component treatment system 22, while enabling the flow paths 44 of the cassette main body 40 to be deformed, makes it possible to set the plurality of paths that are formed by the tubes 16 more easily, and can enhance working efficiency.

The frame 50A retains the cassette main body 40 at the side portions 56, and exposes both surfaces of the cassette main body 40 (i.e., bottom and top surfaces of the cassette main body 40), and the biological component treatment device 14 includes the lid 110 for covering the cassette main body 40 in a state with the biological component cassette 10A arranged therein. In accordance with such features, in the biological component treatment system 22, the weight of the biological component cassette 10A can be reduced, together with making it easy to set the biological component cassette 10A in the biological component treatment device 14.

Specific details were given in the description to provide a thorough understanding of the embodiments. However, it will be understood by one of ordinary skill in the art that the embodiments may be practiced without these specific details. In other instances, well-known circuits, processes, algorithms, structures, and techniques may be shown without unnecessary detail in order to avoid obscuring the embodiments. While illustrative embodiments of the disclosure have been described in detail herein, it is to be understood that the inventive concepts may be otherwise variously embodied and employed, and that the appended claims are intended to be construed to include such variations, except as limited by the prior art.

It should be appreciated that inventive concepts cover any embodiment in combination with any one or more other embodiment, any one or more of the features disclosed herein, any one or more of the features as substantially disclosed herein, any one or more of the features as substantially disclosed herein in combination with any one or more other features as substantially disclosed herein, any one of the aspects/features/embodiments in combination with any one or more other aspects/features/embodiments, use of any one or more of the embodiments or features as disclosed herein. It is to be appreciated that any feature described herein can be claimed in combination with any other feature(s) as described herein, regardless of whether the features come from the same described embodiment.

The above description provides embodiments only, and is not intended to limit the scope, applicability, or configuration of the claims. Rather, the description provides those skilled in the art with an enabling description for implementing the described embodiments. It being understood that various changes may be made in the function and arrangement of elements without departing from the spirit and scope of the appended claims. It will be appreciated from the description, and for reasons of computational efficiency, that the components of the system can be arranged at any appropriate location within a distributed network of components without impacting the operation of the system. Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and this disclosure.

As used herein, the phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together. Various aspects of the present disclosure are described herein with reference to drawings that may be schematic illustrations of idealized configurations.

As used herein, the singular forms “a,” “an,” and “the” are intended to include the plural forms as well, unless the context clearly indicates otherwise. It will be further understood that the terms “include,” “including,” “includes,” “comprise,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof. The term “and/or” includes any and all combinations of one or more of the associated listed items.

Embodiments of the present disclosure may be configured as follows:

(1) A biological component cassette that allows a liquid containing a biological component to flow, the biological component cassette comprising:
a cassette main body having a flow path for the liquid in an interior of the cassette main body, the cassette main body being flexible and having a sheet-like shape; and
a frame that is less flexible than the cassette main body;
wherein the frame includes an accommodation chamber in which the cassette main body is accommodated, wherein a side portion of the frame forms at least part of the accommodation chamber, and
wherein, when the cassette main body is accommodated in the accommodation chamber, one surface of the cassette main body is covered by a bottom portion of the accommodation chamber, and another surface of the cassette main body is exposed to an external environment.

(2) The biological component cassette according to (1), wherein the accommodation chamber comprises a recess in the frame that defines the side portion of the frame and the bottom portion of the frame.

(3) The biological component cassette according to one or more of (1) to (2), wherein the frame includes a cover portion that forms at least part of the bottom portion of the accommodation chamber, and wherein the cover portion includes a support protruding toward the cassette main body.

(4) The biological component cassette according to one or more of (1) to (3), wherein the support contacts the one surface of the cassette main body and supports the cassette main body.

(5) The biological component cassette according to one or more of (1) to (4), wherein:
a detection channel portion is disposed at a predetermined position of the cassette main body through which the flow path extends, wherein the detection channel portion enables an attached device to detect a state of the liquid; and
the support is arranged on the cover portion at a position that opposes the detection channel portion when the cassette main body is accommodated by the accommodation chamber.

(6) The biological component cassette according to one or more of (1) to (5), wherein the detection channel portion protrudes in a thickness direction of the cassette main body, and includes a flat portion extending parallel to the one surface of the cassette main body.

(7) The biological component cassette according to one or more of (1) to (6), wherein:
the support comprises a gripper member that is attached to the cover portion; and
the gripper member includes a flexible portion that is fixable to the cassette main body.

(8) The biological component cassette according to one or more of (1) to (7), wherein:
an outer edge of the cassette main body includes a connector in fluid communication with the flow path; and
the side portion includes an engaging portion that engages with the connector.

(9) The biological component cassette according to one or more of (1) to (8), wherein the engaging portion includes an engagement recess that engages with the connector.

(10) The biological component cassette according to one or more of (1) to (9), wherein:
the connector comprises a flange that projects radially outward from a cylindrical portion of the connector connected to the cassette main body; and
the engaging portion comprises a movement limiter that makes the connector immovable in an axial direction of the connector by accommodating the flange therein.

(11) The biological component cassette according to one or more of (1) to (10), wherein the cassette main body has a rectangular shape in a plan view, and wherein the connector is one of a plurality of connectors provided on four outer edges of the cassette main body, wherein the engaging portion is one of a plurality of engaging portions provided on the side portion of the frame, and wherein each connector is engaged with a corresponding engaging portion when the cassette main body is accommodated by the accommodation chamber.

(12) The biological component cassette according to one or more of (1) to (11), wherein the cassette main body includes a vinyl chloride resin, a polyolefin resin, or a polyurethane resin.

(13) A biological component kit having a tube that allows a liquid containing a biological component to flow, and a biological component cassette to which the tube is connected, wherein the biological component cassette comprises:
a cassette main body having a flow path for the liquid in an interior of the cassette main body, is the cassette main body being flexible and having a sheet-like shape; and
a frame that is less flexible than the cassette main body;
wherein the frame includes an accommodation chamber in which the cassette main body is accommodated, wherein a side portion of the frame forms at least part of the accommodation chamber, and
wherein, when the cassette main body is accommodated in the accommodation chamber, one surface of the cassette main body is covered by a bottom portion of the accommodation chamber, and another surface of the cassette main body is exposed to an external environment.

(14) A biological component treatment system comprising:
a biological component kit having a tube that allows a liquid containing a biological component to flow, and a biological component cassette to which the tube is connected; and
a biological component treatment device that includes a cassette setting location in which the biological component cassette is set;
wherein the biological component cassette comprises:
   a cassette main body having a flow path for the liquid in an interior of the cassette main body, the cassette main body being flexible and having a sheet-like shape; and
   a frame that is less flexible than the cassette main body;
wherein the frame includes an accommodation chamber in which the cassette main body is accommodated, wherein a side portion of the frame forms at least part of the accommodation chamber, and
wherein, when the cassette main body is accommodated in the accommodation chamber, one surface of the cassette main body is covered by at least a portion of the accommodation chamber, and another surface of the cassette main body is exposed to the external environment.

(15) The biological component treatment system according to (14), wherein:
the frame supports the cassette main body at the side portion and exposes two surfaces of the cassette main body to the external environment when supporting the cassette main body; and
the biological component treatment device includes a lid for covering the cassette main body when the biological component cassette is arranged therein.

(16) The biological component treatment system according to one or more of (14) to (15), further comprising:
a load detector that detects pressure of fluid flowing through a detection channel portion of the cassette main body, wherein a cover portion of the frame comprises a protrusion arranged on the cover portion at a position that opposes the detection channel portion when the cassette main body is accommodated by the accommodation chamber.

(17) The biological component treatment system according to one or more of (14) to (16), wherein:
an outer edge of the cassette main body includes a connector in fluid communication with the flow path; and
the side portion of the frame includes an engaging portion that engages with the connector.

(18) The biological component treatment system according to one or more of (14) to (17), wherein the engaging portion includes a recess that engages with the connector.

(19) The biological component treatment system according to one or more of (14) to (18), wherein:
the connector comprises a flange that projects radially outward from the connector; and
the side portion of the frame further comprises a slot-like structure that accommodates the flange to make the connector immovable in an axial direction of the connector.

(20) The biological component treatment system according to one or more of (14) to (19), wherein the slot-like structure is further inward on the side portion of the frame than the recess.

What is claimed is:

1. A biological component cassette that allows a liquid containing a biological component to flow, the biological component cassette comprising:
a cassette main body having a flow path for the liquid in an interior of the cassette main body, the cassette main body being flexible and having a sheet-like shape; and
a frame that is less flexible than the cassette main body;
wherein the frame includes an accommodation chamber in which the cassette main body is accommodated, wherein a side portion of the frame forms at least part of the accommodation chamber, and
wherein, when the cassette main body is accommodated in the accommodation chamber, one surface of the cassette main body is covered by a bottom portion of the accommodation chamber, and another surface of the cassette main body is exposed to an external environment.

2. The biological component cassette according to claim 1, wherein the accommodation chamber comprises a recess in the frame that defines the side portion of the frame and the bottom portion of the frame.

3. The biological component cassette according to claim 1, wherein the frame includes a cover portion that forms at least part of the bottom portion of the accommodation chamber, and wherein the cover portion includes a support protruding toward the cassette main body.

4. The biological component cassette according to claim 3, wherein the support contacts the one surface of the cassette main body and supports the cassette main body.

5. The biological component cassette according to claim 4, wherein:
a detection channel portion is disposed at a predetermined position of the cassette main body through which the flow path extends, wherein the detection channel portion enables an attached device to detect a state of the liquid; and
the support is arranged on the cover portion at a position that opposes the detection channel portion when the cassette main body is accommodated by the accommodation chamber.

6. The biological component cassette according to claim 5, wherein the detection channel portion protrudes in a thickness direction of the cassette main body, and includes a flat portion extending parallel to the one surface of the cassette main body.

7. The biological component cassette according to claim 4, wherein:
the support comprises a gripper member that is attached to the cover portion; and
the gripper member includes a flexible portion that is fixable to the cassette main body.

8. The biological component cassette according to claim 1, wherein:
an outer edge of the cassette main body includes a connector in fluid communication with the flow path; and
the side portion includes an engaging portion that engages with the connector.

9. The biological component cassette according to claim 8, wherein the engaging portion includes an engagement recess that engages with the connector.

10. The biological component cassette according to claim 8, wherein:
the connector comprises a flange that projects radially outward from a cylindrical portion of the connector connected to the cassette main body; and
the engaging portion comprises a movement limiter that makes the connector immovable in an axial direction of the connector by accommodating the flange therein.

11. The biological component cassette according to claim 8, wherein the cassette main body has a rectangular shape in a plan view, and wherein the connector is one of a plurality of connectors provided on four outer edges of the cassette main body, wherein the engaging portion is one of a plurality of engaging portions provided on the side portion of the frame, and wherein each connector is engaged with a corresponding engaging portion when the cassette main body is accommodated by the accommodation chamber.

12. The biological component cassette according to claim 1, wherein the cassette main body includes a vinyl chloride resin, a polyolefin resin, or a polyurethane resin.

13. A biological component kit having a tube that allows a liquid containing a biological component to flow, and a biological component cassette to which the tube is connected, wherein the biological component cassette comprises:
a cassette main body having a flow path for the liquid in an interior of the cassette main body, is the cassette main body being flexible and having a sheet-like shape; and
a frame that is less flexible than the cassette main body;
wherein the frame includes an accommodation chamber in which the cassette main body is accommodated, wherein a side portion of the frame forms at least part of the accommodation chamber, and
wherein, when the cassette main body is accommodated in the accommodation chamber, one surface of the cassette main body is covered by a bottom portion of the accommodation chamber, and another surface of the cassette main body is exposed to an external environment.

14. A biological component treatment system comprising:
a biological component kit having a tube that allows a liquid containing a biological component to flow, and a biological component cassette to which the tube is connected; and
a biological component treatment device that includes a cassette setting location in which the biological component cassette is set;
wherein the biological component cassette comprises:
a cassette main body having a flow path for the liquid in an interior of the cassette main body, the cassette main body being flexible and having a sheet-like shape; and
a frame that is less flexible than the cassette main body;
wherein the frame includes an accommodation chamber in which the cassette main body is accommodated, wherein a side portion of the frame forms at least part of the accommodation chamber, and
wherein, when the cassette main body is accommodated in the accommodation chamber, one surface of the cassette main body is covered by at least a portion of the accommodation chamber, and another surface of the cassette main body is exposed to the external environment.

15. The biological component treatment system according to claim 14, wherein:
the frame supports the cassette main body at the side portion and exposes two surfaces of the cassette main body to the external environment when supporting the cassette main body; and
the biological component treatment device includes a lid for covering the cassette main body when the biological component cassette is arranged therein.

16. The biological component treatment system according to claim 14, further comprising:
a load detector that detects pressure of fluid flowing through a detection channel portion of the cassette main body, wherein a cover portion of the frame comprises a protrusion arranged on the cover portion at a position that opposes the detection channel portion when the cassette main body is accommodated by the accommodation chamber.

17. The biological component treatment system according to claim 14, wherein:
an outer edge of the cassette main body includes a connector in fluid communication with the flow path; and
the side portion of the frame includes an engaging portion that engages with the connector.

18. The biological component treatment system according to claim 17, wherein the engaging portion includes a recess that engages with the connector.

19. The biological component treatment system according to claim 18, wherein:
the connector comprises a flange that projects radially outward from the connector; and
the side portion of the frame further comprises a slot-like structure that accommodates the flange to make the connector immovable in an axial direction of the connector.

20. The biological component treatment system according to claim 19, wherein the slot-like structure is further inward on the side portion of the frame than the recess.
